(19) United States
(12) Patent Application Publication
(10) Pub. No.: US 2022/0056416 A1
(43) Pub. Date: Feb. 24, 2022

(54) RAPID AND EFFICIENT METHOD FOR EXPANDING HUMAN MESENCHYMAL STEM CELLS IN VITRO AND APPLICATION THEREOF

(71) Applicant: Affiliated Hospital of Zunyi Medical University, Zunyi City (CN)

(72) Inventors: Jianhui Xiao, Zunyi City (CN); Yi Luo, Zunyi City (CN); Jianjiang Zhong, Zunyi City (CN); Changyin Yu, Zunyi City (CN)

(73) Assignee: Affiliated Hospital of Zunyi Medical University, Zunyi City (CN)

(21) Appl. No.: 17/340,046

(22) Filed: Jun. 6, 2021

(30) Foreign Application Priority Data
Aug. 21, 2020 (CN) 202010846476.2

(51) Int. Cl. C12N 5/0775

(52) U.S. Cl. CPC C12N 5/0665; C12N 5/0668; C12N 2502/1157; C12N 2502/1164; C12N 2502/1107; C12N 2502/1114; C12N 2502/1192

(57) ABSTRACT

Disclosed is a rapid and efficient method for expanding a human mesenchymal stem cells in vitro. In the method, immune cells in human peripheral blood and human mesenchymal stem cells are co-cultured at a cell number ratio of 1:1 to 400:1, which can significantly enhance the expansion ability of human mesenchymal stem cells; the number of expansion in vitro is more than ten times that of commonly used methods, and the expanded cells still maintain the biological characteristics of stem cells while having stronger self-renewal ability and multidirectional differentiation potential. There is no statistical difference in the expansion-promoting effects of the human peripheral blood immune cells from people of different ages on the human mesenchymal stem cells, regardless of age. Subjects use immune cells in autologous peripheral blood to expand human mesenchymal stem cells, which can avoid the risk of immune rejection.

### FIELD OF THE APPLICATION

The present invention belongs to the field of stem cells and regenerative medicine, in particular, to a rapid and efficient method for expanding human mesenchymal stem cells in vitro and an application thereof.

### BACKGROUND TECHNOLOGY

Human mesenchymal stem cells (MSC) have excellent functional characteristics such as multidirectional differentiation, immune regulation, and promotion of tissue regeneration and repair, with the advantages of low immunogenicity and no tumorigenicity, and are considered to be the most promising resource for stem cell therapy for a variety of incurable diseases such as cardiovascular diseases (Micro circulation. 2017; 24(1)), diabetes (Int Immunopharmacol. 2017; 44:191-196), neurological diseases (J Tissue Eng Regen Med. 2013, 7:169-82.) and malignant tumors (Cell Research. 2008; 18: 500-507). As of 2018, more than 700 human MSC treatment clinical trials (https://www.clinicaltrials.gov) have been approved worldwide, involving hundreds of diseases. Since the first human MSC clinical trial was carried out in 1995, there have been no successful cases of large-scale clinical application. The reasons involving major basic scientific issues and key technologies related to the clinical transformation of human MSC are pending. For example, the number and quality of cells are the source, fundamental and basic issues that plague the clinical transformation of human MSC. The number of MSCs derived from human tissues is limited, which is difficult to meet the requirements of the number of cells required for clinical treatment. Therefore, the in vitro expansion of human MSCs has become a hot issue in the field of regenerative medicine. In recent years, with the rapid development of biotechnology, the expansion of human MSCs through 2D and 3D technologies such as improved culture conditions, bioreactors, microcarriers, and scaffolds has basically solved the problem of limited human MSC cells (Nature Biomedical Engineering 2019; 3:90-104). However, in the process of long-term subculture and in vitro expansion of human MSCs, problems such as abnormal cell morphology, changes in gene protein expression profile, and cell aging caused by environmental stress and physiological factors are faced with, leading to the progressive loss of biological characteristics such as human MSC cell surface markers, stem cell markers, immune regulation capabilities, migration and homing capabilities, self-renewal capabilities, and multi-directional differentiation potential, which seriously affects the quality of cells obtained by expansion, thereby profoundly affecting the clinical treatment effect and resulting in the human MSC obtained by the existing in vitro expansion technology being greatly restricted in scientific research and clinical application.

Therefore, scientists have done a lot of research work to improve the quality of human MSC expanded cells in vitro, and formulated a series of in vitro expansion strategies. For example, laser-irradiated pretreatment of culture medium, addition of exogenous cytokines and extracellular matrix (Biomaterials. 2012; 33: 4480-9; FASEB J. 2011; 25: 1474-85), treatment of natural small molecule compounds (Theranostics. 2016; 6:, 1899-1917) and genetic engineering (Nature. 2008; 451:141-6) have been used to maintain or enhance the stem characteristics and function of MSCs after long-term expansion. However, many innate flaws in design of these strategies limit their clinical application. For example, exogenous cytokines and extracellular matrix need to act continuously, leading to low efficiency and high cost; MSCs through genetic recombination have potential risks of mutation or malformation (J Thorac Cardiovasc Surg. 2008; 136: 1388-9). Therefore, there is an urgent need to find a more effective and reliable method to maintain or enhance the functionality and safety of human MSCs expanded in vitro.

Inflammation is the basis of many chronic and degenerative diseases, as well as an important defense response of the body. In addition, a variety of cytokines produced during inflammation can regulate the regeneration process, remove damaged cells and initiate tissue repair, which is an important driving factor for tissue cell regeneration. For example, in the intestine, activated immune cells produce many inflammatory cytokines, including TNF-α, IL-6, IL-10 and IL-17, etc., which can control the regeneration response of intestinal cells by regulating the expansion and differentiation of intestinal stem cells; further, the production of inflammatory cytokines can prevent intestinal mucosal cells from being damaged and repairing necrotizing enterocolitis caused by aplastic disorders (N Engl J Med. 2011, 364:255-64; Cell. 2004, 23; 118: 229-41). This shows that there are inextricable links between inflammation and tissue regeneration. In recent years, a large amount of evidence has shown that the body microenvironment is one of the important factors that determine the clinical efficacy of human MSC. It has been proven that pretreatment of inflammatory factors (TNF-α and IL-1β can promote MSCs to secrete cytokines (IL-1A, RANTES, G-CSF) and enhance their immune regulation ability (Cell Mol Immunol. 2012; 9:473-81; Cytotherapy, 2017; 19: 181-193). The immune regulation ability of MSC is closely related to its stem characteristics; for example, Shuai et al. confirmed that melatonin can effectively prevent the lack of stem characteristics of rat bone marrow MSCs during in vitro expansion, thereby retaining the effect of in vivo immunotherapy (Theranostics. 2016, 6: 1899-1917). This suggests that in the process of MSC expansion and culture in vitro, an appropriate inflammatory microenvironment is a potential measure to reduce the loss of stem characteristics, or even enhance the stem characteristics.

### SUMMARY OF INVENTION

Based on the common problems of insufficient quantity, poor quality, and limited clinical efficacy of human MSCs expanded in vitro, an object of the present invention is to provide a rapid, efficient, safe and operable method for promoting long-term expansion of human mesenchymal stem cells in vitro, so as to obtain high-quality, safe and effective human mesenchymal stem cells.

To achieve the above object, in the present invention, human MSC and human peripheral blood immune cells such as peripheral blood mononuclear cells (PBMC), or peripheral blood monocytes (PBM) and peripheral blood lymphocytes (PBL) seperated from PBMC are co-cultured, and experimental data show that immune cells may promote the expansion of human MSC, wherein the expansion effect lies in that PBL> PBMC>PBM.

The human mesenchymal stem cells according to the present invention are obtained from fresh umbilical cord, amniotic membrane and cord blood of healthy term cesarean women, including umbilical cord mesenchymal stem cells, amniotic membrane mesenchymal stem cells and cord blood mesenchymal stem cells.

For the human peripheral blood immune cells according to the present invention, PBMC is obtained by collecting peripheral blood of the normal physical examination population and using density gradient centrifugation to obtain PBMC, and further, the PBM's characteristic to easily adhere to the plastic culture plate and grow on the wall is used to separate PBM and PBL from PBMC.

In the present invention, the human peripheral blood immune cells PBMC and the human MSCs are co-cultured at a cell number ratio of 1:1 to 400:1, wherein the expansion effect is the best when the ratio is 300:1.

In the present invention, the human peripheral blood immune cells PBMC and the human MSCs are co-cultured in a contact co-culture way and a non-contact co-culture way, wherein the effect is the better when the contact co-culture way is employed.

The source of the human peripheral blood immune cell PBMC according to the present invention is not restricted by age, and the contact co-culture with the human MSCs may achieve good expansion effect, and there is no statistical difference between groups of different ages.

According to the present invention, the human peripheral blood immune cells PBMC and the human MSCs are co-cultured in a contact way for a long time, and the number of cells obtained is more than 10 times that of common culture methods.

According to the present invention, the human peripheral blood immune cells PBMC and the human MSCs are co-cultured in a contact way, which may significantly enhance the expression of the stem characteristic marker Oct4 of the human MSC.

According to the present invention, the human peripheral blood immune cells PBMC and the human MSCs are co-cultured in a contact way, which may significantly improve the proliferation ability of the human MSC, and promote the expression of the proliferating cell nuclear antigen PCNA of the human MSC.

According to the present invention, the human peripheral blood immune cells PBMC and the human MSCs are co-cultured in a contact way, which may enhance the migration ability of the human MSC, wherein in the scratch experiment, the number of migrating cells increases significantly.

According to the present invention, the human peripheral blood immune cells PBMC and the human MSCs are co-cultured in a contact way, which may improve the self-renewal ability of the human MSC, and significantly promote the cloning of the human MSC.

According to the present invention, the human peripheral blood immune cells PBMC and the human MSCs are co-cultured in a contact way, which may improve the multidirectional differentiation ability of the human MSC, including differentiation into osteoblasts, chondrocytes and adipocytes.

According to the present invention, the human peripheral blood immune cells PBMC and the human MSCs are co-cultured in a contact way, resulting in the human MSC with no immune elimination response to the disease model and being safe and effective.

The expanded human MSC of the present invention is used to treat a mouse model of ulcerative colitis, with significant effect and no immune rejection reaction. More importantly, PBMCs derived from donors of different ages are used for co-culture of the human MSCs for in vitro expansion, and there is no statistical difference, suggesting that the autologous peripheral blood immune cells of the subject may be used to expand the MSCs. Therefore, the human MSC cells expanded in vitro using the present invention have high quality and good therapeutic effect; in particular, the expansion by using the subject's own peripheral blood immune cells overcomes the potential risk of immune rejection, is more secure, and has a huge clinical application prospect.

### BRIEF DESCRIPTION OF DRAWINGS

FIG. 1 illustrates the phenotype identification of human MSC (taking human amniotic membrane MSC as an example). (A) flow cytometry to detect surface molecular markers; (B) immunocytochemical staining to detect vimentin and keratin CK19.Here MSC the abbreviation of mesenchymal stem cells.

FIG. 2 illustrates the effects on the expansion ability of the MSC detected by EDU in case where the human PBMC and the human MSC are co-cultured in a contact way at different cell ratios. Here EDU means 5-ethynyl-2′-deoxyuridine; MSC means mesenchymal stem cells; PBMC means peripheral blood mononuclear cells.

FIG. 3 illustrates the effects on the expansion ability of the MSC detected by EDU in case where the human PBMC and the human MSC are co-cultured in different ways (contact way or non-contact way). Here EDU means 5-ethynyl-2′-deoxyuridine; MSC means mesenchymal stem cells; PBMC means peripheral blood mononuclear cells.

FIG. 4 illustrates the effects on the expansion ability of the MSC detected by EDU in case where the PBMC derived from different ages and the human MSC are co-cultured in a contact way. Here EDU means 5-ethynyl-2′-deoxyuridine; MSC means mesenchymal stem cells; PBMC means peripheral blood mononuclear cells.

FIG. 5 illustrates the effects on the expansion ability of the MSC detected by EDU in case where the different immune cells in the human peripheral blood, including PBMC and its peripheral blood mononuclear cells (PBM) and peripheral blood lymphocytes (PBL), and the human MSC are co-cultured in a contact way. Here EDU means 5-ethynyl-2′-deoxyuridine.

FIG. 6 illustrates the effects on the expansion ability of the MSC detected by EDU in case where the PBL and the human MSC from different tissues (umbilical cord) are co-cultured in a contact way. Here EDU means 5-ethynyl-2′-deoxyuridine; MSC means mesenchymal stem cells; PBL means peripheral blood lymphocytes.

FIG. 7 illustrates the effects on the expansion ability of the MSC detected by EDU in case where the PBL and the human MSC from different tissues (umbilical cord blood) are co-cultured in a contact way. Here EDU means 5-ethynyl-2′-deoxyuridine; MSC means mesenchymal stem cells; PBL means peripheral blood lymphocytes.

FIG. 8 illustrates the effects on the number of expanded cells of human MSC in case where the PBL and the human MSC are co-cultured in a contact way for a long time and in a traditional method for 12 days. Here MSC means mesenchymal stem cells; PBL means peripheral blood lymphocytes.

FIG. 9 illustrates the effects on the stem factor Oct4 in the human MSC and the expression of the proliferating cell nuclear antigen PCNA detected by western blotting in case where the PBL and the human MSC are co-cultured in a contact way. Here MSC means mesenchymal stem cells; PBL means peripheral blood lymphocytes.

FIG. 10 illustrates the effects on the human MSC clone formation in case where the PBL and the human MSC are co-cultured in a contact way and in a traditional method for 9 days. Here MSC means mesenchymal stem cells; PBL means peripheral blood lymphocytes.

FIG. 11 illustrates the effects on the differentiation to osteoblasts, chondrocytes and adipocytes for the human MSC in case where the PBL and the human MSC are co-cultured in a contact way and in a traditional method. Here MSC means mesenchymal stem cells; PBL means peripheral blood lymphocytes.

FIG. 12 illustrates the effects on the migration ability for the human MSC detected by wound healing assay in case where the PBL and the human MSC are co-cultured in a contact way and in a traditional method. Here MSC means mesenchymal stem cells; PBL means peripheral blood lymphocytes.

FIG. 13 illustrates the evaluation of the efficacy of MSC transplantation in mice with ulcerative colitis in case where the PBL and the human MSC are co-cultured in a contact way and in a traditional method. (A) mouse body weight change; (B) mouse disease activity index (DAI) score; (C) pathological observation; (D) pathological score. Here MSC means mesenchymal stem cells; PBL means peripheral blood lymphocytes.

### DETAIL DESCRIPTIONS OF EMBODIMENTS

In order to more fully explain the implementation of the present invention, and that the objectives, technical schemes and advantages of the present invention will become more apparent, the technical solutions of the present invention will be described in more detail with reference to the drawings and examples above. MSCs from different tissues have no effect on the results (see Embodiment 7), and the present embodiment mainly uses human amniotic membrane MSC to illustrate. It should be understood that the specific embodiments described herein are only for illustrating but not for limiting the present invention. In addition, the embodiment of the present invention passed the review of the ethics committee of the Affiliated Hospital of Zunyi Medical College (ethics review number: KLLY-2017-003).

Based on the relationship between inflammation and tissue regeneration, and the influence of inflammatory cytokines on the characteristics and functions of MSC, we use human MSC to co-culture with immune cells in human peripheral blood, including peripheral blood mononuclear cells (PBMC), as well as peripheral blood monocytes (PBM) and peripheral blood lymphocytes (PBL) isolated from PBMC, finding that these immune cells, especially PBL, may significantly promote the expansion of human MSC, and the biological characteristics of the expanded cells have not changed, and even the stemnesss characteristics, homing and migration ability, proliferation ability, self-renewal and multidirectional differentiation ability have been significantly enhanced.

### Embodiment 1: Isolation, Culture and Identification of Human MSC

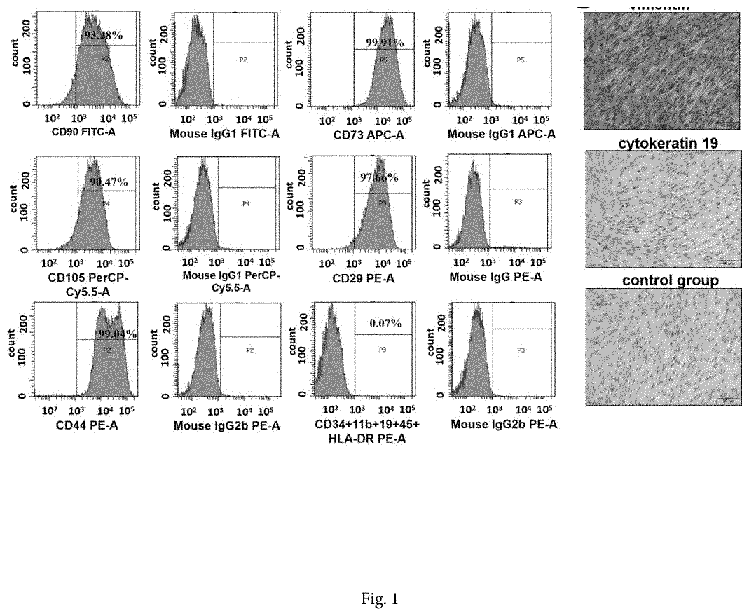

The amniotic membrane tissue is stripped from the fresh placenta of healthy term cesarean section, and the residual blood stains and mucus are repeatedly washed with D-PBS solution containing 1% bi-antibody (final concentration including penicillin of 100 IU/mL, and streptomycin of 100 IU/mL; freshly prepared before use). After cutting the amniotic membrane to pieces each with a size of about 1 cm², divided into 50 mL centrifuge tubes respectively, and then 0.05% trypsin digestion solution containing 0.02% EDTA-2Na that is about 2 times the volume of the amniotic membrane tissue is added for digesting in a constant temperature water bath at 37° C. at 185 rpm for about 30 minutes, filtering with 300 mesh stainless steel filter, and removing the supernatant; then, the above steps are repeated. The digested amniotic membrane is washed with D-PBS containing 1% double antibody once, and an equal volume of 0.5 mg/mL type II collagenase digestion solution containing 0.05 mg/mL DNase I is added for rotating and digesting at 190 rpm at 37° C. for 1 to 1.5 h until the amniotic membrane fragments are completely digested into flocculent, followed by filtering with a 300 mesh filter and collecting cell filtrate for centrifuging at 1500 rpm for 10 minutes; then, the supernatant is removed, and the cell pellet is the human amnion-derived mesenchymal stem cells (hA-MSC). The cells are resuspended in low-glucose DMEM complete medium containing 10% FBS and 10 ng/mL basic fibroblast growth factor (bFGF), plated in 75-cm² cell culture flasks, cultured at a constant temperature with 5% CO2 and 85-100% air saturated humidity at 37° C., and subcultured when the confluence of the cells reaches 80% or more, so as to collect the passage 3 (P3) cells for experiments. In addition, human umbilical cord MSCs are isolated from fresh human umbilical cord tissue by tissue block adhesion method, and human umbilical cord blood MSCs are isolated from fresh human umbilical cord blood by density gradient centrifugation method. The obtained MSCs highly express CD90, CD105, CD73, CD44 and CD29 and other mesenchymal cell surface molecules, do not express hematopoietic stem cell markers such as CD34, CD11b, CD19, CD45 and HLA-DR and MHC class II cell surface molecules (FIG. 1A). The results of immunocytochemical staining show that the cells highly express vimentin, a mesenchymal cell marker, but do not express cytokeratin 19 (CK19), a marker of epithelial cells (FIG. 1B). They have a typical mesenchymal cell phenotype.

### Embodiment 2: Isolation of Human Peripheral Blood Immune Cells

Fresh peripheral blood from a normal physical examination population is taken for diluting it with an equal volume of sterile D-PBS. An appropriate amount of Histopaque-1077 is added to a 15 mL centrifuge tube, and an equal volume of sterile D-PBS diluted blood is slowly added along the tube wall for centrifuging at 2000 rpm for 20 min; the middle albuginea layer is extracted, and an equal volume of sterile D-PBS is added for centrifuging at 1500 rpm for 10 min; washing is performed repeatedly with sterile D-PBS once, and the supernatant is discarded; the cell pellet is suspended in DMEM medium containing 10% FBS and low glucose to obtain PBMC. With the characteristics of peripheral blood mononuclear cells (PBM) that are easy to adhere to and grow on plastic cell culture plates, the PBM and peripheral blood lymphocytes (PBL) are separated from the PBMC isolated above.

### Embodiment 3 Effects on the Expansion Ability of the Human MSC Detected by EDU in Case where the Human PBMC and the Human MSC are Co-Cultured in a Contact Way at Different Cell Ratios

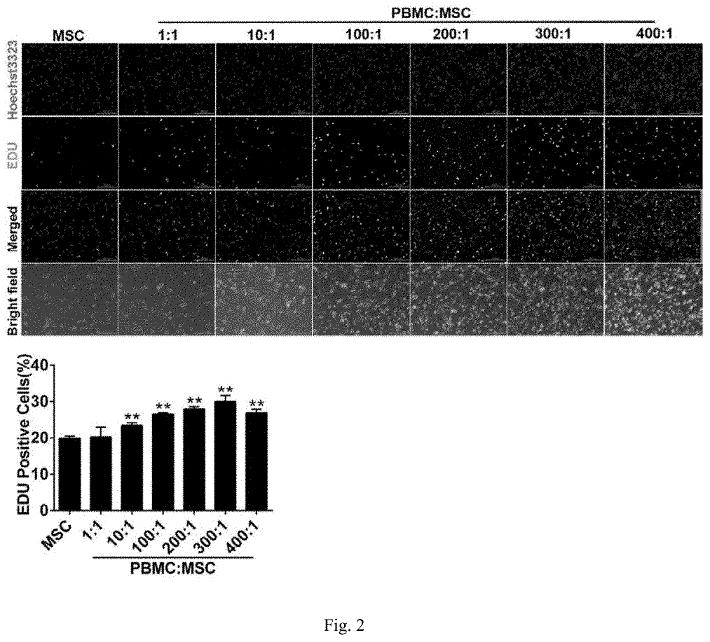

The P3 of human amniotic membrane MSC in the logarithmic growth phase is taken for inoculating on a 24-well cell culture plate at a density of 3×10³/well, and the freshly-isolated PBMC is added after 16 h, wherein the density of PBMC is 3×10³, 3×10⁴, 3×10⁵, 6×10⁵, 9×10⁵ and 1.2×10⁶/well, and the PBMC and the MSC are co-cultured at cell ratios of 1:1, 10:1, 100:1, 200:1, 300:1 and 400:1. After the PBMC and the MSC are co-cultured for 48h, the expansion ability of the MSC is detected by EDU. The results are shown in FIG. 2 and Table 1. The EDU positive cell rate in the normal control MSC group (hereinafter referred to as the “MSC group”) cultured by conventional methods ranges from (19.93±0.63)%; when PBMC:MSC=1:1 or 10:1, the expansion ability of MSC is not significantly enhanced; when PBMC:MSC=100:1, the EDU positive cell rate in MSC increases from (19.93±0.63)% to (26.55±0.37))%; when PBMC:MSC=300:1, the EDU positive cell rate reaches (30.01±1.67%), which is the maximum value; however, when the cell ratio continues to increase, the expansion ability of MSC begins to decrease, e.g., the EDU positive cell rate is (26.89±0.97)% when PBMC:MSC=400:1. Therefore, when the PBMC and the MSC are co-cultured at a cell ratio of 100:1 to 400:1, the expansion ability of the MSC may be significantly improved, wherein when the two are co-cultured at a cell ratio of 300:1, the human MSC has the strongest expansion ability and the best promoting effect on expansion.

TABLE 1
Effects on the expansion ability of the MSC detected
by EDU in case where the contact co-culture is
performed at different cell ratios

| Cell ratios (PBMC:MSC) | EDU positive cell rate (%) |
|---|---|
| 1:1 | 20.25 ± 2.78 |
| 10:1 | 23.43 ± 0.75** |
| 100:1 | 26.55 ± 0.37** |
| 200:1 | 27.93 ± 0.69** |
| 300:1 | 30.01 ± 1.67** |
| 400:1 | 26.89 ± 0.97** |

Note:
The EDU positive cell rate in MSC group is (19.93 ± 0.63)%. Compared with MSC, **p < 0.01. Here EDU means 5-ethynyl-2′-deoxyuridine; MSC means mesenchymal stem cells; PBMC means peripheral blood mononuclear cells.

### Embodiment 4 Effects on the Expansion Ability of the MSC in Case where the Human PBMC and the Human MSC are Co-Cultured in Different Ways (Contact Way or Non-Contact Way)

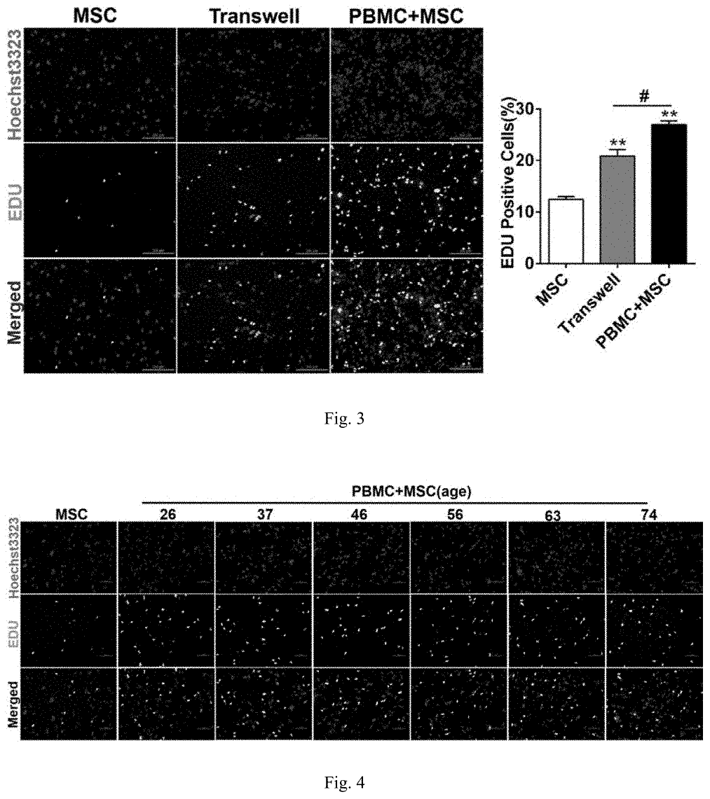

The P3 of human amniotic membrane MSC in the logarithmic growth phase is inoculated into the upper chamber of Transwell (Corning, 3470) at a density of 10³/well, and 10⁵ freshly-separated PBMCs in the lower or upper chambers to be co-cultured with MSCs; after 48 hours, the upper chamber is taken out to test the expansion ability of MSC detected by EDU. The results are shown in FIG. 3, the EDU positive cell rate in the normal control MSC group is (12.43±1.02)%; both the contact and non-contact co-culture of PBMC and MSC may significantly improve the expansion ability of MSC, but the contact way has the better effects, resulting in the EDU positive cell rate of (26.97±1.22)% (Table 2).

TABLE 2
Effects on the expansion ability of the MSC detected
by EDU in case where co-cultured in different
ways (contact way and non-contact way).

| co-culture way of cells | EDU positive cell rate (%) |
|---|---|
| non-contact co-culture | 20.84 ± 2.23* |
| (Transwell) | |
| contact co-culture | 26.97 ± 1.22** |
| (PBMC + MSC) | |

Note:
The EDU positive cell number in MSC group is (12.43 ± 1.02)%. Compared with MSC, **p < 0.01. Here EDU means 5-ethynyl-2′-deoxyuridine; MSC means mesenchymal stem cells; PBMC means peripheral blood mononuclear cells.

### Embodiment 5 Effects of PBMC from Different Donor Ages on the Expansion of Human MSC

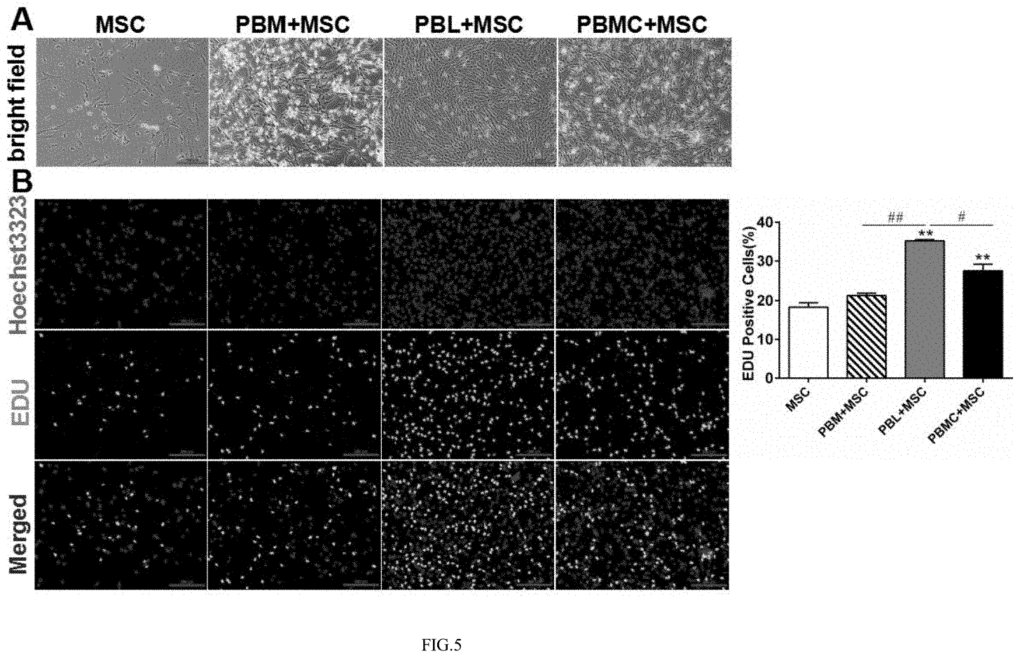

The P3 human amniotic membrane MSCs in the logarithmic growth phase are seeded on a 24-well cell culture plate at a density of 3×10³/well, and the freshly-separated PBMCs from different age groups are added after 16 h. After the PBMC and the MSC are co-cultured for 48 h, the expansion ability of the MSC is detected by EDU. The results (FIG. 4) show that the EDU positive cell rate in the normal control MSC group is (19.82±3.58)%, and compared with the MSC group, the PBMCs derived from different donor ages may significantly enhance the expansion ability of MSCs with an average range of the EDU positive cell rate between (25.54±3.08)% and (26.89±5.48)% (Table 3), but the PBMCs from different donor ages have no statistical difference in the expansion effect of MSC.

TABLE 3
Effects on the expansion ability of the MSC detected by EDU
in case where the PBMC derived from different donor ages and
the human MSC are co-cultured in a contact way.

| age group (years) | number of cases | EDU positive cell rate (%) |
|---|---|---|
| 21-30 | 10 | 25.54 ± 3.08** |
| 31-40 | 10 | 26.80 ± 4.96** |
| 41-50 | 10 | 26.89 ± 5.48** |
| 51-60 | 10 | 26.76 ± 5.53** |
| 61-70 | 10 | 26.80 ± 3.44** |
| 71-80 | 10 | 26.54 ± 4.20** |

Note:
The EDU positive cell number in MSC group is (19.82 ± 3.58)%. Compared with MSC, **p < 0.01. Here EDU means 5-ethynyl-2′-deoxyuridine; MSC means mesenchymal stem cells; PBMC means peripheral blood mononuclear cells.

### Embodiment 6 Effects on the Expansion Ability of the MSC in Case where Different Immune Cells Derived from Human Peripheral Blood and the Human MSC are Co-Cultured in a Contact Way

Density gradient centrifugation method is used to separate PBMC from normal human peripheral blood, and seeded on a 24-well cell culture plate at a density of 3×10⁵/well; after 2h, with the characteristics of PBM that it is easy to adhere to the plastic culture plate to grow on the wall, the PBM and the PBL are separated from the PBMC. In addition, the third generation of human amniotic membrane MSCs in the logarithmic growth phase are inoculated at a density of 3×10³/well into 24-well cell culture plates which containing PBMC, PBM and PBL respectively for co-culturing for 48h, then the morphological characteristics of MSCs are observed under a microscope, and the effects of PBMC, PBM and PBL on the expansion of MSCs are detected by EDU. The results show that after PBMC, PBM, PBL and MSC are co-cultured, MSCs all grew in a long spindle-shaped spiral, but in terms of the cell density, PBL> PBMC>PBM (FIG. 5A). The EDU experiment shows that the normal control MSC group is (18.31±2.01)%, and the co-culture of PBMC, PBM, PBL and MSC may increase the proportion of EDU positive cells to varying degrees with a range from (21.31±1.04) % to (35.21±0.51) % (Table 4); in terms of the ability of promoting expansion, PBL> PBMC>PBM, wherein the PBL has the best effects (FIG. 5B).

TABLE 4
Effects on the expansion ability of the MSC in case where different
immune cells derived from human peripheral blood and the human
MSC are co-cultured in a contact way

| groups | EDU positive cell rate (%) |
|---|---|
| normal control MSC group | 18.31 ± 2.01 |
| monocyte co-culture group | 21.31 ± 1.04 |
| (PBM + MSC) | |
| lymphocyte co-culture group | 35.21 ± 0.51**^{#} |
| (PBL + MSC) | |
| mononuclear cell co-culture group | 27.50 ± 1.10** |
| (PBMC + MSC) | |

Note:
Compared with the MSC group, **p < 0.01; compared with the PBMC + MSC, ^{#}p < 0.05. Here EDU means 5-ethynyl-2′-deoxyuridine; MSC means mesenchymal stem cells; PBMC means peripheral blood mononuclear cells; PBM means peripheral blood mononuclear cells; PBL means peripheral blood lymphocytes.

### Embodiment 7 Effects on the Expansion Ability of the MSC Detected by EDU in Case where the PBL and the MSC from Different Tissue Sources (Umbilical Cord and Umbilical Cord Blood) are Co-Cultured in a Contact Way

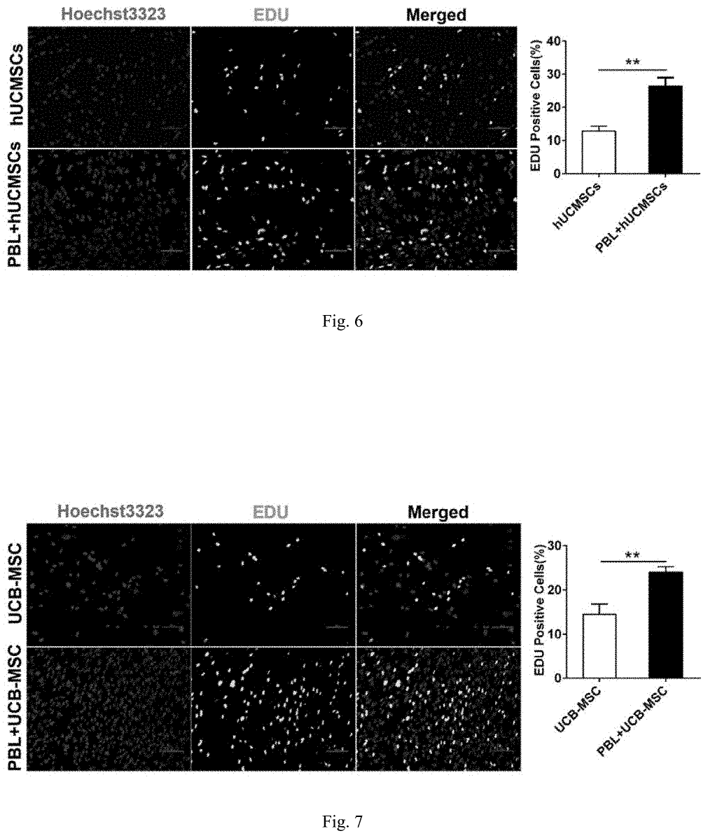

The human umbilical cord-derived mesenchymal stem cells (hUC-MSC) are separated from fresh umbilical cord tissue using the tissue block adhesion method, and the human umbilical cord blood-derived mesenchymal stem cells (hUCB-MSC) are separated from fresh cord blood by the density gradient centrifugation; all are purified by trypsin, and the third generation cells are collected for experiments. The P3 hUC-MSC and hUCB-MSC in the logarithmic growth phase is taken for inoculating in a 24-well cell culture plate at a density of 3×10³/well, and the freshly-separated PBL is added after 16h for co-culturing for 48h; then, EDU is used to detect the expansion ability of hUC-MSC and hUCB-MSC. The results show that similar to hA-MSC derived from PBL and human amniotic membrane tissue (FIG. 5), the contact co-culture of PBL and hUCMSC may significantly promote the expansion of hUC-MSC (FIG. 6); similarly, the contact co-culture of PBL and hUCB-MSC may also significantly promote the expansion of hUCB-MSC (FIG. 7). Therefore, the human peripheral blood immune cells PBL may enhance the expansion ability of MSCs through the contact co-culture, which is not limited by the tissue source of MSCs, and has a significant effect in promoting expansion on umbilical cord tissue and blood-derived MSCs. The following mainly takes hA-MSC as an example to carry out PBL co-culture to evaluate its biological characteristics and functions.

### Embodiment 8 Effects on the Expansion Effect of MSC in Case where the PBL and the MSC are Co-Cultured in the Contact Way In Vitro for a Long Time

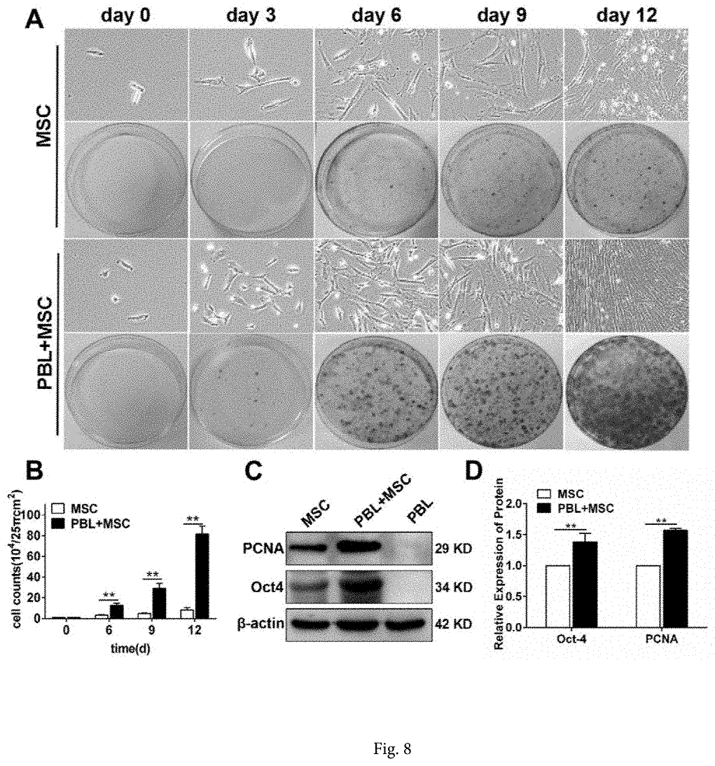

The P3 of human amniotic membrane MSCs in logarithmic growth phase are taken for inoculating in cell culture dishes with diameter of 10 cm at a density of 10⁴ per dish respectively, and the freshly-separately PBL is added after 16h for crystal violet staining and photographing on the 3rd, 6th, 9th and 12th day of the co-culture respectively; on the 6th, 9th and 12th day of the co-culture, the cells are digested with trypsin and centrifuged to collect the cells, and the cells are counted with a cell counter respectively; on the 12th day of co-culture, the total cell protein is extracted, and Western blotting is used to detect the expression levels of the proliferating cell nuclear antigen PCNA and the stem characteristic transcription factor Oct4. The results show that compared with the expansion of MSC alone, when PBL is co-cultured and expanded with MSC for 3 days, the number of MSC cells begin to increase significantly, about 4 times on the 6th day, more than 7 times on the 9th day, and more than 10 times on the 12th day (Table 5, FIG. 8A, B); in addition, when PBL and MSC are co-cultured for 12 d, the expression levels of PCNA and stem characteristic transcription factor Oct4 in MSC are significantly increased (FIGS. 8C, D).

TABLE 5
Effects on the expanded cell number of MSC in case where the
PBL and the MSC are co-cultured in the contact way in vitro
for a long time

| groups | time (day) | cell number (×10⁴) |
|---|---|---|
| MSC | 0 | 1 |
| | 6 | 3.17 ± 0.63 |
| | 9 | 4.92 ± 0.38 |
| | 12 | 8.17 ± 2.47 |
| PBL + MSC | 0 | 1 |
| | 6 | 12.83 ± 2.08** |
| | 9 | 29.17 ± 4.73** |
| | 12 | 81.67 ± 7.64** |

Note:
Compared with MSC, **p < 0.01. Here MSC means mesenchymal stem cells; PBL means peripheral blood lymphocytes.

### Embodiment 9 Effects on the Proliferating Cell Nuclear Antigen PCNA and the Stem Characteristic Transcription Factor Oct4 of MSC in Case where the PBL and the MSC are Co-Cultured in a Contact Way

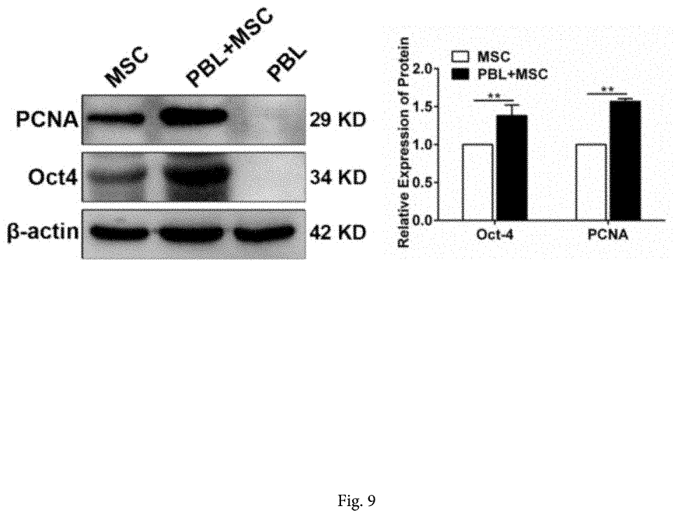

The P3 of human amniotic membrane MSCs in logarithmic growth phase are taken for inoculating in a cell culture dish with a diameter of 10 cm at a density of 2×10⁵, and the freshly-separately PBL is added after 16 h for co-culturing for 48 h; then the total cell protein is extracted, and Western blotting is used to analyze the expression of the proliferating cell nuclear antigen PCNA and the stem characteristic transcription factor Oct4. The results (FIG. 9) show that after the MSC and the PBL are co-cultured, the expression level of the proliferating cell nuclear antigen PCNA and the stem characteristic transcription factor Oct4 of MSC may be significantly enhanced.

### Embodiment 10 Effects on the Self-Renewal Ability of MSC in Case where the PBL and the MSC are Co-Cultured in the Contact Way

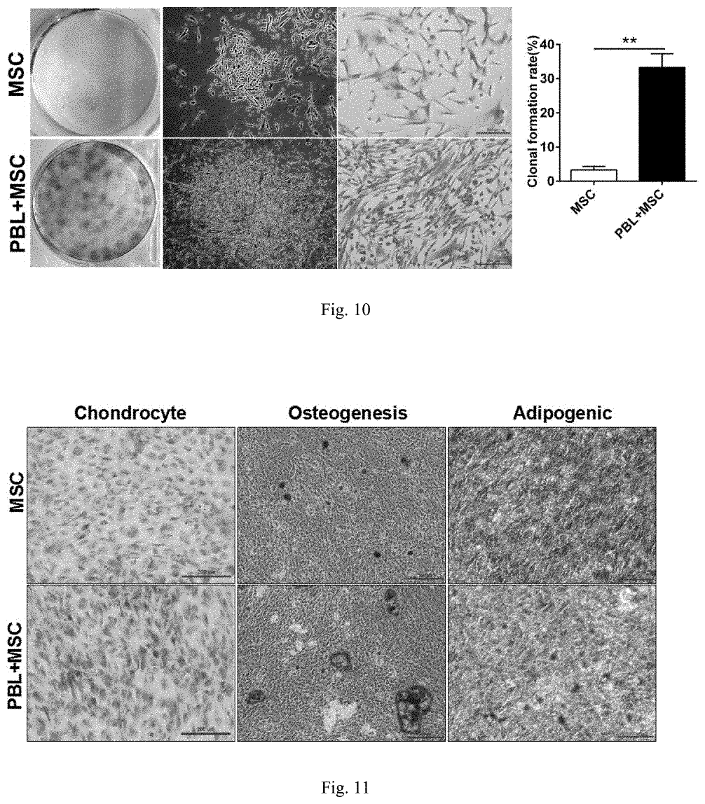

The P3 human amniotic membrane MSC in the logarithmic growth phase is taken for inoculating in a 6-well cell culture plate at a density of 100 cells/well, and the freshly-separated PBL is added after 16h for co-culturing for 9 days; then, the crystal violet staining is used to observe the formation of cell colonies, and photographs are taken and recorded. The results (FIG. 10) show that after 9 days of co-culture of MSC and PBL, the colony formation rate of MSC is 33.3%, which is more than 10 times higher than that of the conventional culture control group (3.3%).

### Embodiment 11 Effects on the Differentiation Ability of MSC in Case where the PBL and the MSC are Co-Cultured in the Contact Way

The P3 human amniotic membrane MSC in the logarithmic growth phase is taken for inoculating in a 6-well cell culture plate at a density of 2×10⁴ cells/well, and the freshly-separated PBL is added after 16h for co-culturing for 48h; when the cell confluence reaches 80%, replace with osteogenic, adipogenic, and chondrogenic differentiation medium. During the whole process of induction and differentiation, the medium is changed every 3 days, and on 21th day, toluidine blue staining is used to detect the production of glycosaminoglycans in the extracellular matrix of chondrogenic differentiation, alizarin red S staining is used to determine the formation of osteogenic differentiation calcified nodules, and oil red O staining is used to determine the formation of adipogenic lipid droplets. The results (FIG. 11) show that compared with MSCs obtained by conventional culture, the MSCs obtained by co-culturing PBL and MSC in a contact way have stronger cartilage matrix glycosaminoglycan production ability, calcium nodule formation ability and lipid droplet production ability. It is suggested that the contact co-culture of PBL and MSC has the ability to enhance MSC differentiation into osteogenic, adipogenic and chondrogenic differentiation.

### Embodiment 12 Effects on the Migration Ability of MSC in Case where the PBL and the MSC are Co-Cultured in the Contact Way

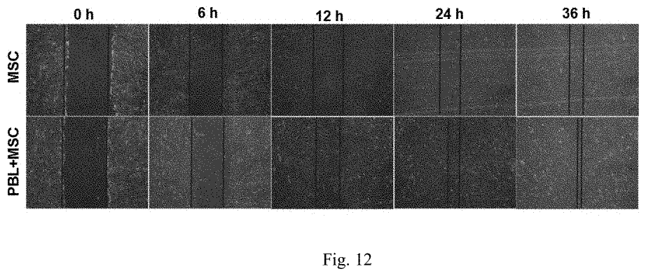

The P3 human amniotic membrane MSC in the logarithmic growth phase is taken for inoculating in a 6-well cell culture plate at a density of 5×10⁴ cells/well, and the freshly-separated PBL is added after 16h for co-culturing until the cell confluence reaches 100%, followed by scoring the culture plate with 200 μL sterile pipette tip, washing away cell debris with sterile PBS, and placing in a 37° C., 5% CO2 incubator with saturated humidity; then, the same scratch position is photographed and recorded at 0, 6, 12, 24, and 36 h of co-culture. The results are shown in FIG. 12, and the contact co-culture of the PBL and the MSC may significantly enhance the migration ability of MSC.

### Embodiment 13 Effects on the Therapeutic Effect of MSC in Case where the PBL and the MSC are Co-Cultured in the Contact Way

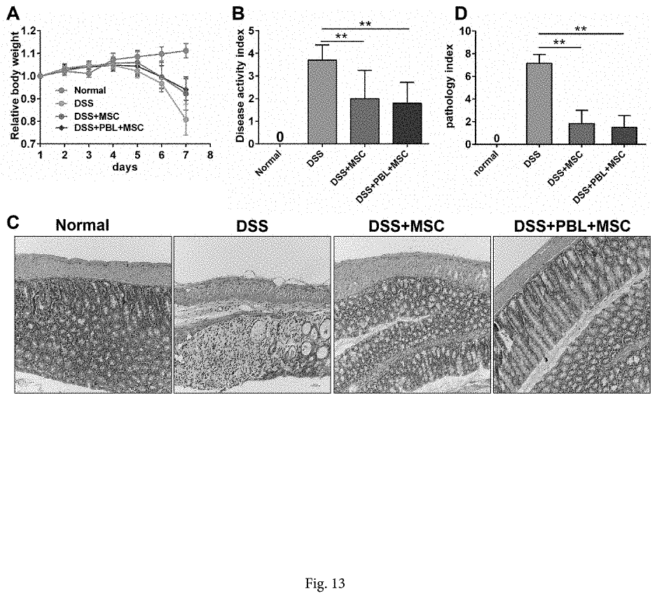

A mouse model of ulcerative colitis is constructed by free diet 3% DSS, and C57 mice aged 5-7 weeks are randomly divided into 4 groups, a total of 40 mice, 10 mice in each group, which are marked with Normal, DSS, DSS+ MSC and DSS+ PBL+ MSC respectively. The DSS+ MSC group and the DSS+ PBL+ MSC group are treated with P4 and lymphocytes by intraperitoneal injection of P4 MSCs (1×10⁷/head) after 48 hours at the start of feeding DSS. Normal and DSS mice are injected with equal volume of sterile PBS at the same time. During the observation, the weight is regularly measured and the death situation is recorded every day, as well as observing whether there are loose stools, bloody stools, etc. The disease activity index (DAI) is calculated based on the weight, diarrhea, blood in the stool, and death of the mouse. Ten days after injection of human amniotic membrane MSC, the mice are sacrificed, and the colon segment is fixed with 4% paraformaldehyde overnight at room temperature, embedded in paraffin, and cut into 4 μm sections for HE staining. The inflammatory cell infiltration and crypt and goblet cell structure of colon tissue are observed under a microscope, and histopathological score is performed according to the previously reported method. The results show (FIG. 13) that on the 5th day of feeding DSS, the mouse begin to show severe inflammatory colitis symptoms, including weight loss, loose stools, bloody stools, etc., and die on the 7th day. Histological analysis showes (FIGS. 13C and D) that DSS feeding causes severe inflammation and damage to the colonic mucosa of mice. After intravenous injection of P4 MSC obtained by co-culture of PBL and P3 MSC, similar to intravenous injection of P4 MSC without immune cell treatment, no immune exclusion reaction occurs, which may significantly reduce DSS-induced weight loss, bloody stool, colon inflammation and damage, and effectively prevent the death of colitis rats (FIG. 13), suggesting that the MSCs obtained by co-culture and expansion of PBL and MSCs are safe and effective for the treatment of the disease model.

1. A rapid and efficient method for expanding human mesenchymal stem cells in vitro, wherein the immune cells of human peripheral blood are employed to co-culture with human mesenchymal stem cells.

2. The rapid and efficient method for expanding human mesenchymal stem cells in vitro according to claim 1, wherein the immune cells of human peripheral blood comprise human peripheral blood mononuclear cells, human peripheral blood monocytes and human peripheral blood lymphocytes.

3. The rapid and efficient method for expanding human mesenchymal stem cells in vitro according to claim 2, wherein the best immune cells of human peripheral blood are the human peripheral blood lymphocytes.

4. The rapid and efficient method for expanding human mesenchymal stem cells in vitro according to claim 1, wherein the method of co-culturing human peripheral blood immune cells with human mesenchymal stem cells comprises co-culturing in contact mode and co-culturing in non-contact mode.

5. The rapid and efficient method for expanding human mesenchymal stem cells in vitro according to claim 4, wherein the method of co-culturing human peripheral blood immune cells with human mesenchymal stem cells is co-culturing in contact mode.

6. The rapid and efficient method for expanding human mesenchymal stem cells in vitro according to claim 1, wherein the immune cells of human peripheral blood are not restricted by an age of a donor.

7. The rapid and efficient method for expanding human mesenchymal stem cells in vitro according to claim 4, wherein the immune cells of human peripheral blood and the human mesenchymal stem cells are co-cultured at a cell number ratio of 1:1 to 400:1.

8. The rapid and efficient method for expanding human mesenchymal stem cells in vitro according to claim 7, wherein the immune cells of human peripheral blood and the human mesenchymal stem cells are co-cultured at a cell number ratio of 300:1.

9. The rapid and efficient method for expanding human mesenchymal stem cells in vitro according to claim 1, wherein the immune cells of human peripheral blood may maintain the biological characteristics of the human mesenchymal stem cells and promote the expression of stem cell markers and a proliferating cell nuclear antigen, significantly enhancing abilities of migration, expansion, self-renewal and differentiation in the daughter cells of human mesenchymal stem cells.

10. The rapid and efficient method for expanding human mesenchymal stem cells in vitro according to claim 1, wherein the method may meet the needs of personalized treatment, which patients can use autologous immune cells of peripheral blood to expand the human mesenchymal stem cells, and the obtained daughter cells of human mesenchymal stem cells are safer for patients them-selves.

11. The rapid and efficient method for expanding human mesenchymal stem cells in vitro according to claim 10, wherein the immune cells of human peripheral blood comprise human peripheral blood mononuclear cells, human peripheral blood monocytes and human peripheral blood lymphocytes.

12. The rapid and efficient method for expanding human mesenchymal stem cells in vitro according to claim 11, wherein the immune cells of human peripheral blood are the human peripheral blood lymphocytes.

13. The rapid and efficient method for expanding human mesenchymal stem cells in vitro according to claim 10, wherein the method of co-culturing human peripheral blood immune cells with human mesenchymal stem cells comprises co-culturing in contact mode and co-culturing in non-contact mode.

14. The rapid and efficient method for expanding human mesenchymal stem cells in vitro according to claim 13, wherein the method of co-culturing human peripheral blood immune cells with human mesenchymal stem cells is co-culturing in contact mode.

15. The rapid and efficient method for expanding human mesenchymal stem cells in vitro according to claim 1, wherein the human peripheral blood immune cells are not restricted by an age of a donor.

16. The rapid and efficient method for expanding human mesenchymal stem cells in vitro according to claim 13, wherein the immune cells in human peripheral blood and the human mesenchymal stem cells are co-cultured at a cell number ratio of 1:1 to 400:1.

17. The rapid and efficient method for expanding human mesenchymal stem cells in vitro according to claim 16, wherein the immune cells of human peripheral blood and the human mesenchymal stem cells are co-cultured at a cell number ratio of 300:1.

18. The rapid and efficient method for expanding human mesenchymal stem cells in vitro according to claim 1, wherein the human peripheral blood immune cells may maintain the biological characteristics of the human mesenchymal stem cells and promote the expression of stem cell markers and a proliferating cell nuclear antigen, significantly enhancing abilities of migration, expansion, self-renewal and differentiation.
